SEQUENCE LISTING (4 sequences)

SEQ ID NO: 1 - DNA, 6 bp, Artificial Sequence
  EcoRI Restriction Target Site
gaattc

SEQ ID NO: 2 - DNA, 23 bp, Artificial Sequence
  Cas9 Target Site
nnnnnnnnnnnnnnnnnnnnngg

SEQ ID NO: 3 - DNA, 23 bp, Artificial Sequence
  S. pyogenes Cas9 Target Site
nnnnnnnnnnnnnnnnnnnnngg

SEQ ID NO: 4 - DNA, 27 bp, Artificial Sequence
  S. thermophilus Cas9 Target Site
nnnnnnnnnnnnnnnnnnnnnnagaan

(19) United States
(12) Patent Application Publication
(10) Pub. No.: US 2017/0020922 A1
(43) Pub. Date: Jan. 26, 2017

(54) GENE EDITING FOR IMMUNOLOGICAL DESTRUCTION OF NEOPLASIA

(71) Applicant: Batu Biologics Inc., San Diego, CA (US)

(72) Inventors: Samuel C. Wagner, San Diego, CA (US); Thomas Ichim, San Diego, CA (US)

(21) Appl. No.: 15/203,378

(22) Filed: Jul. 6, 2016

Related U.S. Application Data
(60) Provisional application No. 62/193,444, filed on Jul. 16, 2015.

(51) Int. Cl. A61K 35/17; C12N 9/22; A61K 45/06; C12N 15/85

(52) U.S. Cl. CPC A61K 35/17; C12N 15/85; C12N 9/22; C12Y 301/00; A61K 45/06; A61K 48/00

(57) ABSTRACT

Disclosed are methods, protocols, and compositions of matter useful for induction and/or propagation of antitumor immune responses through gene editing of immunocytes. Stimulation of antitumor adaptive immunity is achieved through gene editing of autologous or allogeneic lymphocytes in a manner to derepress neoplasia induced suppression. The method can include targets of gene editing disclosed in the current invention include the E3 ubiquitin ligase Cbl-b, CTLA-4, PD-1, TIM-3, killer inhibitory receptor (KIR) and LAG-3.

### RELATED APPLICATIONS

This application is an international application that claims the benefit of priority to U.S. Provisional Patent Application No. 62/193444 filed Jul. 16, 2015, the disclosure of which is incorporated by reference herein in its entirety.

### FIELD OF THE DISCLOSURE

The disclosure pertains to the field of cancer immunotherapy, more specifically, the invention pertains to the utilization of permanent genomic alteration of lymphocytes through deletion at the level of DNA, more specifically, the invention relates to the field of gene editing as applied to immunology of cancer. Methods for increasing the efficiency of a therapy in a subject in need are also contemplated. The methods can include, for example, administering to a subject in a need a therapeutic dose of a genetically altered lymphocyte or a composition of genetically altered lymphocytes.

### BACKGROUND

In recent years the age-old debate of whether cancer is recognized by the immune system has not only been substantially ended, but has also led to therapeutic interventions that have withstood the scrutiny of double blind, placebo controlled trials. Indeed, evidence of immunological control of neoplasia has come in many forms, ranging from animal studies in which the incidence of spontaneous cancer is substantially higher in mice lacking natural killer (NK) cell activity [1-4], to studies in which patients with higher tumor infiltrating lymphocytes possess longer survival [5-7]. Indeed it appears that in the cancer patient a “battle” is actually occurring between tumor-induced immune suppressive mediators and immune responses attempting to clear the tumor from the host. For example, it is widely known that tumors induce the de nova generation of T regulatory cells. The natural function of these cells is to inhibit pathological autoimmunity. During development of self-tolerance in the thymus, while conventional T cells are negatively deleted upon recognition of self-antigen, T regulatory cells that recognize self-antigen are positively selected and promoted to expand by the body [8-10]. The fundamental importance of T regulatory cells is observed in animals lacking T regulatory cells through deletion of FoxP3, in which spontaneous multi-organ autoimmunity occurs, which is also observed in patients possessing a mutation in the gene encoding for the human homologue [11]. In cancer, tumors reprogram the immune system to generate T regulatory cells that serve to protect the tumor against immunological attack. Some examples of this will be listed below.

Jie et al. examined patients with head and neck cancer treated with the anti-EGFR antibody cetuximab. The frequency, immunosuppressive phenotype, and activation status of Treg and NK cells were analyzed in the circulation and tumor microenvironment of cetuximab-treated patients. The antibody treatment increased the frequency of CD4(+)FOXP3(+) intratumoral T regulatory cells. These T regulatory cells suppressed cetuximab-mediated antibody-dependent cellular cytotoxicity (ADCC) and their presence correlated with poor clinical outcome in two prospective clinical trial cohorts [12].

Hanakawa et al. examined 34 patients with tongue cancer immunohistochemically for CD4, CD8, and Forkhead box P3 (Foxp3). Immunoreactive cells were counted in cancer stroma and nest regions, and relationships between cell numbers and disease-free survival rates were analyzed. They found by univariate analysis for disease-free survival that high-level infiltration of Tregs (CD4(+)Foxp3+) into both cancer nests and stroma and the presence of helper T (CD4(+)Foxp3−) cells in cancer stroma as potential predictors of significantly worse prognosis. In early-stage cases (stage I/II), high-level infiltration of Tregs in cancer nests correlated significantly with poor disease-free survival rate [13].

Kim et al. studied 72 patients with early stage (I to III) breast cancer and found increased number of Foxp3(+) Tregs was significantly correlated with tumors with lymph node metastasis (P=0.027), immunopositivity for p53 (P=0.026), and positive for Ki-67 (P<0.001). There were significant correlations between the increased Foxp3(+) Treg/CD4(+) T-cell ratio and lymph node metastasis (P=0.011), the expression of ER (P=0.023), and immunopositivity of p53 (P=0.031) and Ki-67 (P=0.003). Of note, lower Foxp3(+) Treg/CD4(+) T-cell ratio was significantly associated with triple-negative breast cancer (P=0.004) [14].

Numerous other studies have reported similar results showing that Treg cells play a protective role in blocking immune mediated killing of tumors. Specific mechanisms by which Treg cells inhibit conventional T cells include production of the immune suppressive cytokine TGF-beta and interleukin 10. Both of these cytokines act at the level of the naive T cell programming to differentiate into additional Treg cells. Indeed this transfer of tolerogenic capacity was described in the early days of immunology as “infectious tolerance” and studies demonstrated ability to transfer infectious tolerance from mouse to mouse, protecting against various types of autoimmune conditions as well as promoting transplant rejection.

A means of overcoming immune suppression in cancer is by blocking inhibitory signals generated by the tumor, or generated by cells programmed by the tumor. In essence, all T cells possess costimulatory receptors, such as CD40, CD80 and CD86, which are also known as “signal 2”. In this context, Signal 1 is the MHC-antigen signal binding to the T cell receptor, whereas signal 2 provides a costimulatory signal to allow for the T cells to produce autocrine IL-2 and differentiate into effector and memory T cells. When T cells are activated in the absence of signal 2 they become anergic or differentiate into Treg cells. The costimulatory signals exist as a failsafe mechanism to prevent unwanted activation of T cells in absence of inflammation. Indeed, most of the inflammatory conditions associated with pathogens are known to elicit signal 2. For example, viral infections activate toll like receptor (TLR)-3, 7, and 8. Activation of these receptors allows for maturation of plasmacytoid dendritic cells which on the one hand produce interferon alpha, which upregulates CD80 and CD86 on nearby cells, and more directly, the activation of these TLRs results in the plasmacytoid dendritic cell upregulating costimulatory signals. In the case of Gram negative bacteria, upregulation of signal 2 is mediated by LPS binding to TLR-4 which causes direct maturation of myeloid dendritic cells and thus expression of CD40, CD80 and CD86, as well as production of cytokines such as IL-12 and TNF-alpha, which stimulate nearby cells to upregulate signal 2.

Once immune responses have reached their peak, coinhibitory receptors start to become upregulated in order to suppress an immune response that has already performed its function. This is evidenced by upregulation of coinhibitory molecules on T cells such as CTLA4, PD-1, TIM-3, and LAG-3. The finding of co-inhibitory receptors has led to development of antibodies against these receptors, which by blocking their function allow for potent immune responses to ensure unrestrained. The advantage of inhibiting these “immunological checkpoints” is that they not only allow for T cell activation to continue and to not be inhibited by Treg cells, but they also allow for the T cell receptor to become more promiscuous. By this mechanism T cells start attacking various targets that they were not programmed initially to attack.

With the currently approved checkpoint inhibitors, which block CTLA-4 and PD-1, great clinical progress has been achieved in comparison to previously available treatments. In the example of CTLA-4 inhibition, ipilimumab has been approved by regulators and tremelimumab is in advanced stages of clinical trials. Although these anti-CTLA-4 antibodies have modest response rates in the range of 10%, ipilimumab significantly improves overall survival, with a subset of patients experiencing long-term survival benefit. In a phase III trial, tremelimumab was not associated with an improvement in overall survival. Across clinical trials, survival for ipilimumab-treated patients begins to separate from those patients treated in control arms at around 4-6 months, and improved survival rates are seen at 1, 2, and 3 years. Further, in aggregating data for patients treated with ipilimumab, it appears that there may be a plateau in survival at approximately 3 years. Thereafter, patients who remain alive at 3 years may experience a persistent long-term survival benefit, including some patients who have been followed for up to 10 years.

In the case of PD-1 inhibition, Herbst et al. [15] evaluated the single-agent safety, activity and associated biomarkers of PD-L1 inhibition using the MPDL3280A, a humanized monoclonal anti-PD-L1 antibody administered by intravenous infusion every 3 weeks (q3w) to patients with locally advanced or metastatic solid tumors or leukemias. Across multiple cancer types, responses as per RECIST v1.1 were observed in patients with tumors expressing relatively high levels of PD-L1, particularly when PD-L1 was expressed by tumor-infiltrating immune cells. Specimens were scored as immunohistochemistry 0, 1, 2, or 3 if <1%, ≧1% but <5%, ≧5% but <10%, or ≧10% of cells per area were PD-L1 positive, respectively. In the 175 efficacy-evaluable patients, confirmed objective responses were observed in 32 of 175 (18%), 11 of 53 (21%), 11 of 43 (26%), 7 of 56 (13%) and 3 of 23 (13%) of patients with all tumor types, non-small cell lung cancer (NSCLC), melanoma, renal cell carcinoma and other tumors (including colorectal cancer, gastric cancer, and head and neck squamous cell carcinoma). Interestingly, a striking correlation of response to MPDL3280A treatment and tumor-infiltrating immune cell PD-L1 expression was observed. In summary, 83% of NSCLC patients with a tumor-infiltrating immune cell IHC score of 3 responded to treatment, whereas 43% of those with IHC 2 only achieved disease stabilization. In contrast, most progressing patients showed a lack of PD-L1 upregulation by either tumor cells or tumor-infiltrating immune cells.

### SUMMARY

Although progress has been made in extending patient's lives, significant hurdles exist in terms of the patients that do not respond to therapy, or where responses are short lived. We overcome these limitations by administering lymphocytes that have been permanently gene edited so as to not succumb to tumor inhibition. Furthermore, in one embodiment of the invention, the lymphocytes that have been gene edited possess a suicide gene, which allows for destruction of the modified lymphocytes should autoimmunity or pathological consequences arise.

In one aspect, a method of treating cancer may include the steps of obtaining a cellular population containing lymphocytes, decreasing the ability of said lymphocytes to transcribe immune suppressive genes, and administering said lymphocytes into a patient suffering from cancer.

In some embodiments, the lymphocytes are substantially purified for T cell content. In some embodiments, the purification for T cell content is achieved by selecting cells for expression of a marker selected from the group including a) CD3; b) CD4; c) CD8; and d) CD90. In some embodiments, the lymphocytes are substantially purified for NK cell content. In some embodiments, the purification for NK cell content is achieved by selecting cells for expression of a marker selected from the group including CD56, CD57, KIR, and CD16. In some embodiments, the gene editing is achieved using one or more zinc finger nucleases. In some embodiments, the gene editing is achieved by intracellularly delivering into said lymphocyte a DNA molecule possessing a specific target sequence and encoding the gene product of said target sequence into a non-naturally occurring Clustered Regularly Interspaced Short Palindromic Repeats associated system comprising one or more vectors comprising a first regulatory element that functions in said lymphocyte and is operably linked to at least one nucleotide sequence encoding a CRISPR-Cas system guide RNA that hybridizes with said target sequence, and a second regulatory element functioning in a lymphocyte that is operably linked to a nucleotide sequence encoding a Type-II Cas9 protein, wherein components (a) and (b) are located on same or different vectors of the system, whereby the guide RNA targets the sequence whose deletion is desired and the Cas9 protein cleaves the DNA molecule, in a manner such that expression of at least one gene product is substantially inhibited, and in a manner that the Cas9 protein and the guide RNA do not naturally occur together.

In some embodiments, the vectors of the system further comprise one or more nuclear localization signals. In some embodiments, the guide RNAs comprise a guide sequence fused to a transactivating er (tracr) sequence. In some embodiments, the Cas9 protein is tailored for maximal activity based on DNA codon for said target gene and said lymphocyte. In some embodiments, the immune suppressive gene is selected from the group including the E3 ubiquitin ligase Cbl-b, CTLA-4, PD-1, TIM-3, killer inhibitory receptor (KIR), LAG-3, CD73, Fas, the aryl hydrocarbon receptor, Smad2, Smad4, TGF-beta receptor, and ILT-3. In some embodiments, the patient is preconditioned with a lymphocyte depleting regimen prior to infusion of said gene edited lymphocytes. In some embodiments, the lymphocytes are autologous to said patient. In some embodiments, the lymphocytes are allogeneic to said patient. In some embodiments, the lymphocytes are chimeric antigen receptor (CAR)-T cells. In some embodiments, the lymphocytes are transfected with a suicide gene. In some embodiments, the suicide gene is thymidylate synthase.

In some embodiments, an orally inducible construct is added to said lymphocytes to allow induction of immune stimulatory genes in a controllable manner. In some embodiments, the lymphocytes are generated from cord blood progenitor cells. In some embodiments, the lymphocytes are one or a plurality of cell lines. In some embodiments, the cell line is NK-92. In some embodiments, the lymphocyte is an innate lymphocyte cell. In some embodiments, the innate lymphoid cells are selected from the group including innate lymphoid cells 1, innate lymphoid cells 2, innate lymphoid cells 3, and lymphoid tissue inducer cells. In some embodiments, the innate lymphoid cells 1 express T bet and respond to IL-12 by secretion of interferon gamma, however lack expression of perform and CD56. In some embodiments, the innate lymphoid cells 2 produce IL-4 and IL-13. In some embodiments, the innate lymphoid cells 3 produce IL-17a and IL-22. In some embodiments, the lymphoid tissue inducer cells are cells involved in the induction of memory T cells. In some embodiments, the T cells are Th1 cells. In some embodiments, the Th1 cells are capable of secreting cytokines selected from the group including interferon gamma, interleukin 2, and TNF-beta.

In some embodiments, the Th1 cells express markers selected from the group including CD4, CD94, CD119 (IFNy R1), CD183 (CXCR3), CD186 (CXCR6), CD191 (CCRI), CD195 (CCR5), CD212 (IL-12R˜1&2), CD254 (RAN KL), CD278 (ICOS), IL-18R, MRP1, NOTCH3, and TIM3. In some embodiments, the lymphocytes are immune cells endowed with anticancer activity by the process of gene editing. In some embodiments, the anticancer activities of said immune cells are ability to directly kill said cancer cells. In some embodiments, the anticancer activities of said immune cells are ability to induce other cells to kill said cancer cells. In some embodiments, the anticancer activities of said immune cells are ability to inhibit proliferation of said cancer cells.

In some embodiments, the anticancer activities of said immune cells are ability to induce other cells to inhibit proliferation of said cancer cells. In some embodiments, the anticancer activities of said immune cells are ability to directly kill blood vessel cells associated with said cancer. In some embodiments, the anticancer activities of said immune cells are ability to induce other immune cells to directly kill blood vessel cells associated with said cancer. In some embodiments, the anticancer activities of said immune cells are ability to directly block proliferation of blood vessel cells associated with said cancer. In some embodiments, the anticancer activities of said immune cells are ability to induce other immune cells to block proliferation of blood vessel cells associated with said cancer.

In some embodiments, a chemotherapeutic agent is utilized to enhance anticancer response. In some embodiments, the chemotherapeutic agent is an alkylating agent. In some embodiments, the alkylating agent is selected from the group including ifosfamide, nimustine hydrochloride, cyclophosphamide, dacarbazine, melphalan, and ranimustine. In some embodiments, the chemotherapeutic agent is an antimetabolite. In some embodiments, the anti-metabolite is selected from the group including gemcitabine hydrochloride, enocitabine, cytarabine ocfosfate, a cytarabine formulation, tegafur/uracil, a tegafur/gimeracil/oteracil potassium mixture, doxifluridine, hydroxycarbamide, fluorouracil, methotrexate, and mercaptopurine.

In some embodiments, the chemotherapeutic agent is an antitumor antibiotic. In some embodiments, the antitumor antibiotic is selected from a group including idarubicin hydrochloride, epirubicin hydrochloride, daunorubicin hydrochloride, daunorubicin citrate, doxorubicin hydrochloride, pirarubicin hydrochloride, bleomycin hydrochloride, peplomycin sulfate, mitoxantrone hydrochloride, and mitomycin C. In some embodiments, the chemotherapeutic agent is an alkaloid. In some embodiments, the alkaloid is selected from a group comprising of etoposide, irinotecan hydrochloride, vinorelbine tartrate, docetaxel hydrate, paclitaxel, vincristine sulfate, vindesine sulfate, and vinblastine sulfate.

In some embodiments, the chemotherapeutic agent is a hormone therapy. In some embodiments, the hormone therapy is selected from a group including anastrozole, tamoxifen citrate, toremifene citrate, bicalutamide, flutamide, and estramustine phosphate. In some embodiments, the chemotherapy is a platinum complex. In some embodiments, the platinum complex is selected from a group comprising of carboplatin, cisplatin, and nedaplatin. In some embodiments, the chemotherapy is an angiogenesis inhibitor. In some embodiments, the angiogenesis inhibitor is selected from a group comprising of: thalidomide, neovastat, and bevacizumab.

In another aspect, a genetically modified lymphocyte is disclosed that includes a first vector, the first vector including a nucleic acid encoding a protein that deletes one or more immune checkpoint genes from the lymphocyte.

In some embodiments, the one or more immune checkpoint genes is selected from the group including E3 ubiquitin ligase Cbl-B, CTLA-4, PD-1, TIM-3, killer inhibitory receptor (KIR), LAG-3, CD73, Fas, aryl hydrocarbon receptor, Smad2, Smad4, TGF-beta receptor, and ILT-3.

In some embodiments, the genetically modified lymphocyte further includes a second vector, the second vector having a nucleic acid encoding a Cas9 endonuclease, and a nucleic acid encoding a CRISPR, wherein the CRISPR is complimentary to at least one immune checkpoint gene in the lymphocyte.

In another aspect, a composition including any one or more of the genetically modified lymphocytes disclosed herein with a carrier or anti-cancer therapeutic.

In another aspect, a method of treating cancer is disclosed that includes administering any one or more of the genetically modified lymphocyte disclosed herein or a composition disclosed herein to a subject in need thereof.

### DETAILED DESCRIPTION OF CERTAIN PREFERRED EMBODIMENTS

Described herein are compositions and methods for gene editing of checkpoint genes. Essentially, the invention teaches the application of gene editing technology as a means of generating lymphocytes resistant to inhibitory signals. Furthermore, the invention teaches the use of suicide genes to allow for deletion of manipulated lymphocytes administered to the host. Means of inducing the process of gene deletion are known in the art. The original notion that gene editing may be feasible was provided by Barrangou et al. [16] who showed that clustered regularly interspaced short palindromic repeats (CRISPR) are found in the genomes of most Bacteria and Archaea and after bacteriophage challenge, the bacteria integrated new spacers derived from phage genomic sequences. Removal or addition of particular spacers modified the phage-resistance phenotype of the cell. They concluded that CRISPR, together with associated cas genes, provided resistance against phages, and resistance specificity is determined by spacer-phage sequence similarity. These techniques, which are incorporated by reference provided a clue that editing or deleting DNA segments may be possible. In 2013, Mali et al. took the observations that bacteria and archaea utilize CRISPR and the CRISPR-associated (Cas) systems, combined with short RNA to direct degradation of foreign nucleic acids, and applied the concept to gene-editing of human cells. They developed a type II bacterial CRISPR system to function with custom guide RNA (gRNA) in human cells. They used the system to delete the human adeno-associated virus integration site 1 (AAVS1). They obtained targeting rates of 10 to 25% in 293T cells, 13 to 8% in K562 cells, and 2 to 4% in induced pluripotent stem cells [17]. Subsequent variations on the theme were reported, which were effective at deleting human genomic DNA, these methods are incorporated by reference [18, 19].

In the description that follows, a number of terms are used extensively. The following definitions are provided to facilitate understanding of the present alternatives.

As used herein, “a” or “an” may mean one or more than one.

As used herein, the term “about” indicates that a value includes the inherent variation of error for the method being employed to determine a value, or the variation that exists among experiments.

“Binding” refers to a sequence-specific, non-covalent interaction between macromolecules. Not all components of a binding interaction need be sequence-specific (e.g., contacts with phosphate residues in a DNA backbone), as long as the interaction as a whole is sequence-specific.

“Binding protein” as described herein is a protein that is able to bind to another molecule. A binding protein can bind to, for example, a DNA molecule (a DNA-binding protein), an RNA molecule (an RNA-binding protein) and/or a protein molecule (a protein-binding protein).

“CRISPR/Cas nuclease” or “CRISPR/Cas nuclease system” includes a non-coding RNA molecule (guide) RNA that binds to DNA and Cas proteins (Cas9) with nuclease functionality (e.g., two nuclease domains). See, e.g., U.S. Provisional Application No. 61/823,689. Collectively, CRISPR system refers to transcripts and other elements involved in the expression of or directing the activity of CRISPR-associated (“Cas”) genes, including sequences encoding a Cas gene, a tracr (trans-activating CRISPR), a tracr-mate sequence (encompassing a “direct repeat” and a tracr RNA-processed partial direct repeat in the context of an endogenous CRISPR system), a guide sequence (also referred to as a “spacer” in the context of an endogenous CRISPR system), or other sequences and transcripts from a CRISPR locus. A sequence or template that may be used for recombination into the targeted locus comprising the target sequences is referred to as an “editing template” or “editing polynucleotide” or “editing sequence.” In aspects of the invention, an exogenous template polynucleotide may be referred to as an editing template. In one embodiment, the recombination is homologous recombination.

“Cleavage” as described herein refers to the breakage of the covalent backbone of a DNA molecule. Cleavage can be initiated by a variety of methods including, but not limited to, enzymatic or chemical hydrolysis of a phosphodiester bond. Both single-stranded cleavage and double stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

“Guide sequence” is any polynucleotide sequence having sufficient complementarity with a target polynucleotide sequence to hybridize with the target sequence and direct sequence-specific binding of a CRISPR complex to the target sequence.

“Sequence” refers to a nucleotide sequence of any length, which can be DNA or RNA; can be linear, circular or branched and can be either single-stranded or double-stranded. The term “donor sequence” refers to a nucleotide sequence that is inserted into a genome.

“Target site” or “target sequence” is a nucleic acid sequence that defines a portion of a nucleic acid to which a binding molecule will bind, provided sufficient conditions for binding exist. For example, the sequence 5′-GAATTC-3′ is a target site for the Eco RI restriction endonuclease.

“Checkpoint genes” as described herein are genes or protein products thereof that inhibit immune responses. Within the context of the invention, checkpoint genes include: a) the E3 ubiquitin ligase Cbl-b; b) CTLA-4; c) PD-1; d) TIM-3; e) killer inhibitory receptor (KIR); f) LAG-3; g) CD73; h) Fas; i) the aryl hydrocarbon receptor; j) Smad2; k) Smad4; l) TGF-beta receptor; and m) ILT-3.

“Programmed cell death protein 1,” or PD-1 is a protein that functions as an immune checkpoint and plays a role in down regulating the immune system by preventing the activation of T cells to reduce autoimmunity and promote self-tolerance. PD-1 has an inhibitory effect of programming apoptosis in antigen specific T cells in the lymph nodes and simultaneously reducing apoptosis in regulatory T cells. PD-1 has two ligands PD-L1 and PD-L2. Binding of PD-L1 to PD-1 allows the transmittal of an inhibitory signal which reduces the proliferation of CD8+ T cells at lymph nodes. PD-L1 can also bind PD-1 on activated T cells, B cells and myeloid cells to modulate activation or inhibition. The upregulation of PD-L1 may also allow cancers to evade the host immune system.

As used herein, “nucleic acid,” “polynucleotide,” and “oligonucleotide” refers to a deoxyribonucleotide or ribonucleotide polymer, in linear or circular conformation, and in either single- or double-stranded form. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone.

A “vector” or “construct” is a nucleic acid used to introduce heterologous nucleic acids into a cell that can also have regulatory elements to provide expression of the heterologous nucleic acids in the cell. Vectors include but are not limited to plasmid, minicircles, yeast, and viral genomes. In some alternatives, the vectors are plasmid, minicircles, viral vectors, DNA or mRNA. In some alternatives, the vector is a lentiviral vector or a retroviral vector. In some alternatives, the vector is a lentiviral vector.

In one embodiment of the invention, a genetically engineered form of (CRISPR)-CRISPR-associated (Cas) protein system [20] of Streptococcus pyogenes is used to induce gene editing of immune checkpoint genes as described for other genes and incorporated by reference [21]. In this system, the type II CRISPR protein Cas9 is directed to genomic target sites by short RNAs, where it functions as an endonuclease. In the naturally occurring system, Cas9 is directed to its DNA target site by two noncoding CRISPR RNAs (crRNAs), including a trans-activating crRNA (tracrRNA) and a precursor crRNA (pre-crRNA). In the synthetically reconstituted system, these two short RNAs can be fused into a single chimeric guide RNA (gRNA). A Cas9 mutant with undetectable endonuclease activity (dCas9) has been targeted to genes in bacteria, yeast, and human cells by gRNAs to silence gene expression through steric hindrance [22].

In one embodiment of the invention, disclosed is the use of a regulatory element that is operably linked to one or more elements of a CRISPR system so as to drive expression of the one or more elements of the CRISPR system, with the goal of manipulating DNA encoding for checkpoint genes in lymphocytes in a manner that prevents lymphocytes from expressing said checkpoint genes. Checkpoint genes relevant for the practice of the invention include: a) the E3 ubiquitin ligase Cbl-b; b) CTLA-4; c) PD-1; d) TIM-3; e) killer inhibitory receptor (KIR); f) LAG-3; g) CD73; h) Fas; i) the aryl hydrocarbon receptor; j) Smad2; k) Smad4; l) TGF-beta receptor; and m) ILT-3. CRISPRs (Clustered Regularly Interspaced Short Palindromic Repeats), also known as SPIDRs (Spacer Interspersed Direct Repeats), constitute a family of DNA loci that are generally unique to a particular bacterial species. The CRISPR locus comprises a distinct class of interspersed short sequence repeats (SS Rs) that were recognized in E. coli [23, 24]. However, the finding of SS Rs is not specific to E. Coli, as other groups have identified them in other bacteria such as in tuberculosis [25]. The CRISPR loci differ from other SS Rs by the structure of the repeats, which are called short regularly spaced repeats (SRSRs) [26]. Repeats of SRSRs are short elements that occur in clusters that are regularly spaced by unique intervening sequences with a substantially constant length. Although the repeat sequences are highly conserved between strains, the number of interspersed repeats and the sequences of the spacer regions typically differ from strain to strain.

In the embodiment of the invention in which an endogenous CRISPR system is utilized to delete immune checkpoint genes, formation of a CRISPR complex (which is made of a guide sequence hybridized to a target sequence and complexed with one or more Cas proteins) will cause cleavage of one or both strands in or near the target sequence. The tracr sequence used for the practice of the invention may comprise or consist of all or a portion of a wild-type tracr sequence, may also form part of a CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of a CRISPR complex. When inducing gene editing in lymphocytes a Cas enzyme, a guide sequence linked to a tracr-mate sequence, and a tracr sequence could each be operably linked to separate regulatory elements on separate vectors. Useful vectors include viral constructs, which are well known in the art, in one preferred embodiment lentiviral constructs are utilized. In one embodiment of the invention, two or more of the elements expressed from the same or different regulatory elements, may be combined in a single vector, with one or more additional vectors providing any components of the CRISPR system not included in the first vector.

In one embodiment of the invention, CRISPR system elements that are combined in a single vector may be arranged in any suitable orientation, such as one element located 5′ with respect to or 3′ with respect to a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence, and a tracr sequence embedded within one or more intron sequences. In some embodiments, the CRISPR enzyme, guide sequence, tracr mate sequence, and tracr sequence are operably linked to and expressed from the same promoter.

In one embodiment of the invention, a vector comprises one or more insertion sites, such as a restriction endonuclease recognition sequence. In some embodiments, one or more insertion sites are located upstream and/or downstream of one or more sequence elements of one or more vectors. In some embodiments, a vector comprises an insertion site upstream of a tracr mate sequence, and optionally downstream of a regulatory element operably linked to the tracr mate sequence, such that following insertion of a guide sequence into the insertion site and upon expression the guide sequence directs sequence-specific binding of a CRISPR complex to a target sequence in a eukaryotic cell. In some embodiments, a vector comprises two or more insertion sites, each insertion site being located between two tracr mate sequences so as to allow insertion of a guide sequence at each site. In such an arrangement, the two or more guide sequences may comprise two or more copies of a single guide sequence, two or more different guide sequences, or combinations of these. When multiple different guide sequences are used, a single expression construct may be used to target CRISPR activity to multiple different, corresponding target sequences within a cell.

In one embodiment, gene deletion of immune checkpoint genes is accomplished using a Cas9 nickase that may be used in combination with guide sequence(s), e.g., two guide sequences, which target respectively sense and anti-sense strands of the DNA target. This combination allows both strands to be nicked and used to induce non-homologous DNA end joining (NHEJ). In a preferred embodiment, an enzyme coding sequence encoding a CRISPR enzyme is codon optimized for expression in lymphocytes. It is known that the predominance of selected tRNAs in a cell is generally a reflection of the codons used most frequently in peptide synthesis. Accordingly, genes can be tailored for optimal gene expression in a given type of lymphocyte based on codon optimization. Codon usage tables are readily available, for example, at the “Codon Usage Database”, and these tables can be adapted in a number of ways [27].

The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art. The guide sequence may be selected to target any target sequence. In some embodiments, the target sequence is a sequence within a genome of a cell. Exemplary target sequences include those that are unique in the target genome. For example, for the S. pyogenes Cas9, a unique target sequence in a genome may include a Cas9 target site of the form MMMMMMMMMNNNNNNNNNNNNXGG where NNNNNNNNNNNXGG (N is A, G, T, or C; and X can be anything) has a single occurrence in the genome. A unique target sequence in a genome may include an S. pyogenes Cas9 target site of the form MMMMMMMMMNNNNNNNNNNXGG where NNNNNNNNNNNNXGG (N is A, G, T, or C; and X can be anything) has a single occurrence in the genome. For the S. thermophilus CRISPR1 Cas9, a unique target sequence in a genome may include a Cas9 target site of the form MMMMMMMMNNNNNNNNNNNNXXAGAAW where NNNNNNNNNNNNXXAGAAW (N is A, G, T, or C; X can be anything; and W is A or T) has a single occurrence in the genome. In some embodiments, a guide sequence is selected to reduce the degree of secondary structure within the guide sequence. Secondary structure may be determined by any suitable polynucleotide folding algorithm. A tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a CRISPR complex at a target sequence, wherein the CRISPR complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence.

Cancer diseases are associated with out of control cell growth. These can be malignant tumors or malignant neoplasmas involving abnormal cell growth, which can invade and spread to other parts of the body.

“Natural killer cells” or NK cells are a type of cytotoxic lymphocyte critical to the innate immune system. The role NK cells play is analogous to that of cytotoxic T cells in the vertebrate adaptive immune response. NK cells provide rapid responses to viral-infected cells and respond to tumor formation. The function of NK cells is critical to the prevention of de novo tumor growth through a process known as immune surveillance (Dunn et al., Cancer immunoediting: from immunosurveillance to tumor escape. Nat Immunol 3, 991-998 (2002); Langers et al., Natural killer cells: role in local tumor growth and metastasis. Biologics: targets & therapy 6, 73-82 (2012); both references incorporated in their entireties herein).

In one embodiment of the invention, NK cells are utilized as the target cell for gene editing. NK cell expansion methods are widely known in the art, for example, in one methodology NK cells are purified by removing T cells from the cell population, after removal of T cells, the remaining cells are cultured in a medium supplemented with 2500 to 3000 IU/mL of IL-2, and transplanting the NK cells which are amplified from the remaining cells to a patient. The method may comprise a step of removing hematopoietic progenitor cells or other cells from the cell population. In the step of transplanting the NK cells to the patient, the gene edited NK cells may be transplanted together with NK cell progenitors, T cells, NKT cells, hematopoietic progenitor cells or the like. One gene that may be edited is the NK KIR gene. In the method for adoptive immunotherapy of the present invention, the step of transplanting the NK cells to the patient may be implemented by a step of administering the pharmaceutical composition of the present invention to the patient.

In the adoptive immunotherapy method of the present invention, the cell population which is comprised of NK cells may be prepared from at least one kind of cell selected from a group consisting of: hematopoietic stem cells derived from any stem cells selected from a group consisting embryonic stem cells, adult stem cells and induced pluripotent stem cells (iPS cells); hematopoietic stem cells derived from umbilical cord blood; hematopoietic stem cells derived from peripheral blood; hematopoietic stem cells derived from bone marrow blood; umbilical cord blood mononuclear cells; and peripheral blood mononuclear cells. The donor of the cell population which is comprised of NK cells may be the recipient, that is, the patient himself or herself, a blood relative of the patient, or a person who is not a blood relative of the patient. The NK cells may be derived from a donor whose major histocompatibility antigen complex (MHC) and killer immunoglobulin-like receptors (KIR) do not match with those of the recipient. The gene editing step may be performed on NK progenitor cells, thus circumventing the need for wide-scale transfection.

In the amplifying stem of the invention the cell population which is comprised of NK cells may be prepared using various procedures known to those skilled in the art. For example, to collect mononuclear cells from blood such as umbilical cord blood and peripheral blood, the buoyant density separation technique may be employed. NK cells may be collected with immunomagnetic beads. Furthermore, the NK cells may be isolated and identified using a FACS (fluorescent activated cell sorter) or a flow cytometer, following immunofluorescent staining with specific antibodies against cell surface markers. The NK cells may be prepared by separating and removing cells expressing cell surface antigens CD3 and/or CD34, with immunomagnetic beads comprising, but not limited to, Dynabeads (trade mark) manufactured by Dynal and sold by Invitrogen (now Life Technologies Corporation), and CliniMACS (trade mark) of Miltenyi Biotec GmbH. T cells and/or hematopoietic progenitor cells may be selectively injured or killed using specific binding partners for T cells and/or hematopoietic progenitor cells. The step of removing the T cells from the mononuclear cells may be a step of removing cells of other cell types, such as hematopoietic progenitor cells, B cells and/or NKT cells, together with the T cells. The step of removing the hematopoietic progenitor cells from the mononuclear cells may be a step of removing cells of other cell types, such as T cells, B cells and/or NKT cells, together with the hematopoietic progenitor cells. In the amplifying method of the present invention, the mononuclear cells separated from the umbilical cord blood and peripheral blood may be cryopreserved and stored to be thawed in time for transplantation to the patient. Alternatively, the mononuclear cells may be frozen during or after amplification by the method for amplifying the NK cells of the present invention, and thawed in time for transplantation to the patient. Any method known to those skilled in the art may be employed in order to freeze and thaw the blood cells. Any commercially available cryopreservation fluid for cells may be used to freeze the cells.

In one embodiment the invention provides a means of generating a population of cells with tumoricidal ability that have been gene edited. 50 mL of peripheral blood is extracted from a cancer patient and peripheral blood monoclear cells (PBMC) are isolated using the Ficoll Method. PBMC are subsequently resuspended in 10 mL STEM-34 media and allowed to adhere onto a plastic surface for 2-4 hours. The adherent cells are then cultured at 37° C. in STEM-34 media supplemented with 1,000 U/mL granulocyte monocyte colony-stimulating factor (GM-CSF) and 500 U/mL IL-4 after non-adherent cells are removed by gentle washing in Hanks Buffered Saline Solution (HBSS). Half of the volume of the GM-CSF and IL-4 supplemented media is changed every other day. Immature DCs are harvested on day 7. In one embodiment, said generated DC are used to stimulate T cell and NK cell tumoricidal activity. Specifically, generated DC may be further purified from culture through use of flow cytometry sorting or magnetic activated cell sorting (MACS), or may be utilized as a semi-pure population. Gene editing may be performed prior to co-culture, during co-culture, or after co-culture. In a preferred embodiment gene editing is performed prior to co-culture. DC may be added into said patient in need of therapy with the concept of stimulating NK and T cell activity in vivo, or in another embodiment may be incubated in vitro with a population of cells containing T cells and/or NK cells. In one embodiment DC are exposed to agents capable of stimulating maturation in vitro. Specific means of stimulating in vitro maturation include culturing DC or DC containing populations with a toll-like receptor (TLR) agonist. Another means of achieving DC maturation involves exposure of DC to TNF-alpha at a concentration of approximately 20 ng/mL. In order to activate T cells and/or NK cells in vitro, cells are cultured in media containing approximately 1000 IU/mL of interferon gamma. Incubation with interferon gamma may be performed for a period of 2 hours to 7 days. Preferably, incubation is performed for approximately 24 hours, after which T cells and/or NK cells are stimulated via the CD3 and CD28 receptors. One means of accomplishing this is by addition of antibodies capable of activating these receptors. In one embodiment approximately, 2 μg/mL of anti-CD3 antibody is added, together with approximately 1 μg/mL anti-CD28. In order to promote survival of T cells and NK cells, was well as to stimulate proliferation, a T cell/NK mitogen may be used. In one embodiment the cytokine IL-2 is utilized. Specific concentrations of IL-2 useful for the practice of the invention are approximately 500 U/mL IL-2. Media containing IL-2 and antibodies may be changed every 48 hours for approximately 8-14 days. In one particular embodiment DC are included to said T cells and/or NK cells in order to endow cytotoxic activity towards tumor cells. In a particular embodiment, inhibitors of caspases are added in the culture so as to reduce the rate of apoptosis of T cells and/or NK cells. Generated cells can be administered to a subject intradermally, intramuscularly, subcutaneously, intraperitoneally, intraarterially, intravenously (including a method performed by an indwelling catheter), intratumorally, or into an afferent lymph vessel. Gene editing means that have utilized transfection of T cells with CRISPR-Cas9 are incorporated by reference [28-32].

In some embodiments, the culture of the cells is performed by starting with purified lymphocyte populations, for example, the step of separating the cell population and cell sub-population containing a T cell can be performed, for example, by fractionation of a mononuclear cell fraction by density gradient centrifugation, or a separation means using the surface marker of the T cell as an index. Subsequently, isolation based on surface markers may be performed. Examples of the surface marker include CD3, CD8 and CD4, and separation methods depending on these surface markers are known in the art. For example, the step can be performed by mixing a carrier such as beads or a culturing container on which an anti-CD8 antibody has been immobilized, with a cell population containing a T cell, and recovering a CDS-positive T cell bound to the carrier. As the beads on which an anti-CD8 antibody has been immobilized, for example, CD8 MicroBeads, Dynabeads M450 CD8, and Eligix anti-CD8 mAb coated nickel particles can be suitably used. This is also the same as in implementation using CD4 as an index and, for example, CD4 MicroBeads, Dynabeads M-450 CD4 can also be used. In some embodiments of the invention, T regulatory cells are depleted before initiation of the culture. Depletion of T regulatory cells may be performed by negative selection by removing cells that express makers such as neuropilin, CD25, CD4, CTLA4, and membrane bound TGF-beta.

Experimentation by one of skill in the art may be performed with different culture conditions in order to generate effector lymphocytes, or cytotoxic cells, that possess both maximal activity in terms of tumor killing, as well as migration to the site of the tumor. For example, the step of culturing the cell population and cell sub-population containing a T cell can be performed by selecting suitable known culturing conditions depending on the cell population. In addition, in the step of stimulating the cell population, known proteins and chemical ingredients, etc., may be added to the medium to perform culturing. For example, cytokines, chemokines or other ingredients may be added to the medium. “Chemokines” as described herein are a family of small cytokines, or signaling proteins secreted by cells. Chemokines can be either basal or inflammatory. Inflammatory chemokines are formed upon inflammatory stimuli such as IL-1, TNF-alpha, LPS or by viruses, and participate in the inflammatory response attracting immune cells to the site of inflammation. Without being limiting, inflammatory chemokines can include CXCL-8, CCL2, CCL3, CCL4, CCL5, CCL11 and CXCL10. In some alternatives, an immune cell comprises a first vector, wherein the first vector comprises a nucleic acid encoding a protein that induces T-cell proliferation and/or induces production of an interleukin, an interferon, a PD-1 checkpoint binding protein, HMGB1, MyD88, a cytokine or a chemokine. In some alternatives, the protein is a T-cell or NK-cell chemokine. In some alternatives, the chemokine is CXCL-8, CCL2, CCL3, CCL4, CCL5, CCL11 or CXCL10. In some alternatives, the chemokine comprises CCL1, CCL2, CCL3, CCR4, CCL5, CCL7, CCL8, CCL11, CCL13, CCL17, CCL22, CCL24, or CCL26. In some alternatives, the chemokine is CCL1, CCL2, CCL3, CCR4, CCL5, CCL7, CCL8/MCP-2, CCL11, CCL13/MCP-4, HCC-1/CCL14, TARC/CCL17, CCL19, CCL22, CCL24, CCL26. CCL27, VEGF, PDGF, lymphotactin (XCL1), Eotaxin, FGF, EGF, IP-10, GCP-2/CXCL6, NAP-2/CXCL7, ITAC/CXCL11, CXCL12, CXCL13 or CXCL15. In some alternatives, the chemokines are selected from a group consisting of EGF, Eotaxin, FGF-2, FLT-3L, Fractalkine, G-CSF, GM-CSF, GRO, IL-10, IL-12(p40), IL-12(p70), IL-13, IL-13, IL-15, Il18A, IL-1RA, Il-1a, IL-1b, Il-2, Il-3, Il-4, Il-5, Il-6, Il-7, IL-8, IL-9, INF-α2, INFγ, IP-10, MCP-1, MCP-3, MDC, MIP-1a, MIP-1b, PDGF-AA, PDGF-BB, RANTES, TGF-α, TGF-β, VEGF, sCD401, 6CKINE, BCA-1, CTACK, ENA78, Eotaxin-2, Eotaxin-3, 1309, IL-16, IL-20, IL-21, IL-23, IL-28a, IL-33, LIF, MCP-2, MCP-4, MIP-1d, SCF, SDF-1atb, TARC, TPO, TRAIL, TSLP, CCL1ra/HCC-1, CCL19/MIP beta, CCL20/MIP alpha, CXCL11/1-TAC, CXCL6/GCP2, CXCL7/NAP2, CXCL9/MIG, IL-11, IL-29/ING-gamma, M-CSF and XCL1/Lymphotactin.

Herein, the cytokine is not particularly limited as far as it can act on the T cell, and examples thereof include IL-2, IFN-gamma, transforming growth factor (TGF)-beta, IL-15, IL-7, IFN-alpha, IL-12, CD40L, and IL-27. From the viewpoint of enhancing cellular immunity, particularly suitably, IL-2, IFN-γ, or IL-12 is used and, from the viewpoint of improvement in survival of a transferred T cell in vivo, IL-7, IL-15 or IL-21 is suitably used. In addition, the chemokine is not particularly limited as far as it acts on the T cell and exhibits migration activity, and examples thereof include RANTES, CCL21, MIP1α, MIP1β, CCL19, CXCL12, IP-10 and MIG. The stimulation of the cell population can be performed by the presence of a ligand for a molecule present on the surface of the T cell, for example, CD3, CD28, or CD44 and/or an antibody to the molecule. Further, the cell population can be stimulated by contacting with other lymphocytes such as antigen presenting cells (dendritic cell) presenting a target peptide such as a peptide derived from a cancer antigen on the surface of a cell. In addition to assessing cytotoxicity and migration as end points, it is within the scope of the current invention to optimize the cellular product based on other means of assessing T cell activity, for example, the function enhancement of the T cell in the method of the present invention can be assessed at a plurality of time points before and after each step using a cytokine assay, an antigen-specific cell assay (tetramer assay), a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer, a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. In vivo assessment of the efficacy of the generated cells using the invention may be assessed in a living body before first administration of the T cell with enhanced function of the present invention, or at various time points after initiation of treatment, using an antigen-specific cell assay, a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

The following citations (and any citation in the present specification) are each expressly incorporated by reference in its entirety.
1. Bryceson, Y. T. and H. G. Ljunggren, Tumor cell recognition by the NK cell activating receptor NKG2D. Eur J Immunol, 2008. 38(11): p. 2957-61.
2. Waldhauer, I. and A. Steinle, NK cells and cancer immunosurveillance. Oncogene, 2008. 27(45): p. 5932-43.
3. Guerra, N., et al., NKG20-deficient mice are defective in tumor surveillance in models of spontaneous malignancy. Immunity, 2008. 28(4): p. 571-80.
4. Guillerey, C., et al., Immunosurveillance and therapy of multiple myeloma are CD226 dependent. J Clin Invest, 2015. 125(5): p. 2077-89.
5. Horn, T., et al., The prognostic effect of tumour-infiltrating lymphocytic subpopulations in bladder cancer. World J Ural, 2015.
6. de Jong, R. A., et al., Presence of tumor-infiltrating lymphocytes is an independent prognostic factor in type I and II endometrial cancer. Gynecol Oncol, 2009. 114(1): p. 105-10.
7. Leffers, N., et al., Prognostic significance of tumor-infiltrating T-lymphocytes in primary and metastatic lesions of advanced stage ovarian cancer. Cancer Immunol Immunother, 2009. 58(3): p. 449-59.
8. Coquet, J. M., et al., Epithelial and dendritic cells in the thymic medulla promote CD4+Foxp3+ regulatory T cell development via the CD27-CD70 pathway. J Exp Med, 2013. 210(4): p. 715-28.
9. Cowan, J. E., et al., The thymic medulla is required for Foxp3+ regulatory but not conventional CD4+ thymocyte development. J Exp Med, 2013. 210(4): p. 675-81.
10. Bautista, J. L., et al., Intraclonal competition limits the fate determination of regulatory T cells in the thymus. Nat Immunol, 2009. 10(6): p. 610-7.
11. Ochs, H. D., E. Gambineri, and T. R. Torgerson, IPEX, FOXP3 and regulatory T-cells: a model for autoimmunity. Immunol Res, 2007. 38(1-3): p. 112-21.
12. Jie, H. B., et al., CTLA-4+ Regulatory TCells Increased in Cetuximab-Treated Head and Neck Cancer Patients Suppress NK Cell Cytotoxicity and Correlate with Poor Prognosis. Cancer Res, 2015. 75(11): p. 2200-10.
13. Hanakawa, H., et al., Regulatory T-cell infiltration in tongue squamous cell carcinoma. Acta Otolaryngol, 2014. 134(8): p. 859-64.
14. Kim, S. T., et al., Tumor-infiltrating lymphocytes, tumor characteristics, and recurrence in patients with early breast cancer. Am J Clin Oneal, 2013. 36(3): p. 224-31.
15. Herbst, R. S., et al., Predictive correlates of response to the anti-PD-L1 antibody MPDL3280A in cancer patients. Nature, 2014. 515(7528): p. 563-7.
16. Barrangou, R., et al., CRISPR provides acquired resistance against viruses in prokaryotes. Science, 2007. 315(5819): p. 1709-12.
17. Mali, P., et al., RNA-guided human genome engineering via Cas9. Science, 2013. 339(6121): p. 823-6.
18. Cho, S. W., et al., Targeted genome engineering in human cells with the Cas9 RNA-guided endonuclease. Nat Biotechnol, 2013. 31(3): p. 230-2.
19. Wang, H., et al., One-step generation of mice carrying mutations in multiple genes by CRISPR/Cas-mediated genome engineering. Cell, 2013. 153(4): p. 910-8.
20. Jinek, M., et al., A programmable dual-RNA-guided DNA endonuclease in adaptive bacterial immunity. Science, 2012. 337(6096): p. 816-21.
21. Cong, L., et al., Multiplex genome engineering using CRISPR/Cas systems. Science, 2013. 339(6121): p. 819-23.
22. Qi, L. S., et al., Repurposing CRISPR as an RNA-guided platform for sequence-specific control of gene expression. Cell, 2013. 152(5): p. 1173-83.
23. Ishino, Y., et al., Nucleotide sequence of the lap gene, responsible for alkaline phosphatase isozyme conversion in Escherichia coli, and identification of the gene product. J Bacterial, 1987. 169(12): p. 5429-33.
24. Nakata, A., M. Amemura, and K. Makino, Unusual nucleotide arrangement with repeated sequences in the Escherichia coli K-12 chromosome. J Bacterial, 1989. 171(6): p. 3553-6.
25. Groenen, P. M., et al., Nature of DNA polymorphism in the direct repeat cluster of Mycobacterium tuberculosis; application for strain differentiation by a novel typing method. Mal Microbial, 1993. 10(5): p. 1057-65.
26. Mojica, F. J., et al., Biological significance of a family of regularly spaced repeats in the genomes of Archaea, Bacteria and mitochondria. Mal Microbial, 2000. 36(1): p. 244-6.
27. Nakamura, Y., T. Gojobori, and T. Ikemura, Codon usage tabulated from the international DNA sequence databases; its status 1999. Nucleic Acids Res, 1999. 27(1): p. 292.
28. Matheson, N. J., A. A. Peden, and P. J. Lehner, Antibody-free magnetic cell sorting of genetically modified primary human CD4+ T cells by one-step streptavidin affinity purification. PLoS One, 2014. 9(10): p. el11437.
29. Meissner, T. B., et al., Genome editing for human gene therapy. Methods Enzymol, 2014. 546: p. 273-95.
30. Ebina, H., et al., A high excision potential of TALENs for integrated DNA of HIV-based lentiviral vector. PLoS One, 2015. 10(3): p. e0120047.
31. Choi, Y. S. and S. Crotty, Retroviral vector expression in TCR transgenic CD4(+) T cells. Methods Mal Biol, 2015. 1291: p. 49-61.
32. Li, C., et al., Inhibition of HIV-1 infection of primary CD4+ T cells by gene editing of CCRS using adenovirus-delivered CRISPR/Cas9. J Gen Viral, 2015.

What is claimed is:

1. A method of treating cancer comprising the steps of:
a) obtaining a cellular population containing lymphocytes;
b) decreasing the ability of said lymphocytes to transcribe immune suppressive genes; and
c) administering said lymphocytes into a patient suffering from cancer.

2. The method of claim 1, wherein said lymphocytes are substantially purified for T cell content by selecting cells for expression of a marker selected from the group consisting of: a) CD3; b) CD4; c) CD8; and d) CD90.

3. The method of claim 1, wherein said lymphocytes are substantially purified for NK cell content by selecting cells for expression of a marker selected from the group consisting of: a) CD56; b) CD57; c) KIR; and d) CD16.

4. The method of claim 1, wherein said gene editing is achieved by intracellularly delivering into said lymphocyte a DNA molecule possessing a specific target sequence and encoding the gene product of said target sequence into a non-naturally occurring Clustered Regularly Interspaced Short Palindromic Repeats associated system comprising one or more vectors comprising:
a) a first regulatory element that functions in said lymphocyte and is operably linked to at least one nucleotide sequence encoding a CRISPR-Cas system guide RNA that hybridizes with said target sequence, and
b) a second regulatory element functioning in a lymphocyte that is operably linked to a nucleotide sequence encoding a Type-II Cas9 protein, wherein components (a) and (b) are located on same or different vectors of the system, whereby the guide RNA targets the sequence whose deletion is desired and the Cas9 protein cleaves the DNA molecule, in a manner such that expression of at least one gene product is substantially inhibited; and in a manner that the Cas9 protein and the guide RNA do not naturally occur together.

5. The method of claim 4, wherein the vectors of the system further comprise one or more nuclear localization signals, wherein said guide RNAs comprise a guide sequence fused to a transactivating er (tracr) sequence, and wherein said Cas9 protein is tailored for maximal activity based on DNA codon for said target gene and said lymphocyte.

6. The method of claim 1, wherein said immune suppressive gene is selected from the group consisting of:
a) the E3 ubiquitin ligase Cbl-b;
b) CTLA-4;
c) PD-1;
d) TIM-3;
e) killer inhibitory receptor (KIR);
f) LAG-3;
g) CD73;
h) Fas;
i) the aryl hydrocarbon receptor;
j) Smad2;
k) Smad4;
l) TGF-beta receptor; and
m) ILT-3.

7. The method of claim 1, further comprising preconditioning the patient with a lymphocyte depleting regimen prior to infusion of said gene edited lymphocytes.

8. The method of claim 1, wherein said lymphocytes are autologous to said patient.

9. The method of claim 1, wherein said lymphocytes are allogeneic to said patient.

10. The method of claim 1, wherein said lymphocytes are chimeric antigen receptor (CAR)-T cells.

11. The method of claim 1, wherein said lymphocytes are transfected with a suicide gene, and wherein said suicide gene is thymidylate synthase.

12. The method of claim 1, further comprising adding an orally inducible construct to the lymphocytes to allow induction of immune stimulatory genes in a controllable manner.

13. The method of claim 1 further comprising generating said lymphocytes from cord blood progenitor cells.

14. The method of claim 1, wherein said lymphocyte is an innate lymphocyte cell selected from the group consisting of:
a) innate lymphoid cells 1;
b) innate lymphoid cells 2;
c) innate lymphoid cells 3; and
d) lymphoid tissue inducer cells.

15. The method of claim 14, wherein said innate lymphoid cells 2 produce IL-4 and IL-13.

16. The method of claim 14, wherein said innate lymphoid cells 3 produce IL-17a and IL-22.

17. The method of claim 1, wherein said lymphocytes are immune cells endowed with anticancer activity by the process of gene editing, wherein said anticancer activities of said immune cells are ability to directly kill said cancer cells, and wherein the anticancer activities include one or more of the following: 1) ability to induce other cells to kill said cancer cells; 2) ability to inhibit proliferation of said cancer cells; 3) ability to induce other cells to inhibit proliferation of said cancer cells; 4) ability to directly kill blood vessel cells associated with said cancer; 5) ability to induce other immune cells to directly kill blood vessel cells associated with said cancer; 6) ability to directly block proliferation of blood vessel cells associated with said cancer; and 7) ability to induce other immune cells to block proliferation of blood vessel cells associated with said cancer.

18. The method of claim 1, further comprising administering a chemotherapeutic agent to enhance anticancer response, wherein said chemotherapeutic agent is an antitumor antibiotic, and wherein said antitumor antibiotic is selected from a group comprising of: idarubicin hydrochloride, epirubicin hydrochloride, daunorubicin hydrochloride, daunorubicin citrate, doxorubicin hydrochloride, pirarubicin hydrochloride, bleomycin hydrochloride, peplomycin sulfate, mitoxantrone hydrochloride, and mitomycin C.

19. A genetically modified lymphocyte comprising a first vector, the first vector comprising a nucleic acid encoding a protein that deletes one or more immune checkpoint genes from the lymphocyte, wherein the one or more immune checkpoint genes is selected from the group consisting of E3 ubiquitin ligase Cbl-B, CTLA-4, PD-1, TIM-3, killer inhibitory receptor (KIR), LAG-3, CD73, Fas, aryl hydrocarbon receptor, Smad2, Smad4, TGF-beta receptor, and ILT-3.

20. The genetically modified lymphocyte of claim 19, further comprising:
a second vector, wherein the second vector comprises a nucleic acid encoding a Cas9 endonuclease; and
a nucleic acid encoding a CRISPR, wherein the CRISPR is complimentary to at least one immune checkpoint gene in the lymphocyte.
